(19) United States
(12) Patent Application Publication
(10) Pub. No.: US 2005/0095281 A1
(43) Pub. Date: May 5, 2005

(54) Liposomal formulations and methods of use

(75) Inventors: Hans Ernst Jan Hofland, Foster City, CA (US); Lisa Cencia Rohan, Pittsburgh, PA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: OPTIME Therapeutics, Inc., Petaluma, CA (US); Megee-Womens Health Corporation, Pittsburgh, PA (US)

(21) Appl. No.: 10/799,922

(22) Filed: Mar. 12, 2004

Related U.S. Application Data
(60) Provisional application No. 60/454,729, filed on Mar. 14, 2003.

(51) Int. Cl. A61K 31/685; A61K 9/127; A61K 31/20

(52) U.S. Cl. 424/450; 514/78; 514/558

(57) ABSTRACT

The present invention provides liposomal formulations comprising a lipid vesicle and at least one single chain lipid active agent. In addition, the present invention provides methods for using the same for preventing an infection in a mammal.

### CROSS-REFERENCES TO RELATED APPLICATIONS

This application claims benefit of U.S. Provisional Application No. 60/454,729, filed Mar. 14, 2003, which application is incorporated herein by reference for all purposes.

### BACKGROUND OF THE INVENTION

Over 65 million people in the U.S. were infected with a sexually transmitted disease (STD) in the year 2000. Centers for Disease Control and Prevention, “Tracking the Hidden Epidemics,” Internet Communication (2000). http://www.cdc.gov/nchstp/dstd/ Stats_Trends/Trends2000.pdf. The most common STD infections include chlamydia, gonorrhea, syphilis, herpes simplex type 2 (HSV-2), human papilloma virus (HPV), hepatitis B, and trichomoniasis. In addition to those already infected, it is estimated that every year in the U.S. approximately 1 million people will contract HSV-2, 5.5 million will contract HPV, 3 million will contract chlamydia, and 40,000 will contract HIV.

The resulting economic toll is staggering: In 1994, the CDC estimated that all STDs combined have an annual cost of approximately $17 billion. These costs include both indirect expenses (associated with lost productivity, job loss) and direct expenses (associated with cost of drugs for treatment, hospitalization, surgery, doctor's fees, etc.). Overall, HIV is by far the most costly of these diseases with an annual price tag of approximately $6.7 billion.

Most common bacterial STD infections, such as gonorrhea, syphilis, chancroid, chlamydia, and trichomoniasis, can be treated with a single or multi-dose regimen of antibiotics or antimicrobials (Kingston et al., “Treatment of sexually transmitted infections with single-dose therapy: a double-edged sword,” Drugs, 62:871-878 (2002)). However, permanent damage, e.g., sterility, often occurs prior to diagnosis and treatment of bacterial STD infections. Moreover, while current antibacterial drugs have a high rate of success, there has been an alarming trend in the resistance of several of these organisms to some antibiotics.

In contrast to bacterial infections, there are no eradicative therapies presently available for viral STDs such as HSV-2, HIV, and HPV. Therapeutic agents have been developed to slow down the course of viral infection or to control viral outbreaks. These include viral DNA polymerase inhibitors as acyclovir, valaciclovir, and famicilovir for the control of genital herpes, and zidovudine, for the inhibition of HIV replication. Snoeck et al., “New treatments for genital herpes,” Curr. Opin. Infect. Dis., 15:49-55 (2002). Surgery and topical application of fluorouracil is used to control HPV, although neither treatment is capable of preventing future outbreaks.

While women appear to be infected with STDs (excluding HIV) at about the same rate as men, they often face a greater health risk stemming from such infections. In many women, STDs are asymptomatic and may not be identified during routine examinations unless a specific test is performed. Without treatment, diseases such as gonorrhea and chlamydia may lead to the development of more serious medical problems including pelvic inflammatory disease, infertility, or ectopic pregnancy. More troubling are HPV (type 16) and HSV-2, both of which have no cure, and both of which have been linked to the development of cervical cancer. Many STDs also lead to complications with pregnancy and delivery. Approximately 30 to 40% of all pre-term births and infant deaths appear to be linked to an STD infection. National Center for HIV, STD and TB Prevention Division of HIV STD Prevention Centers for Disease Control, “HIV/AIDS Among U.S. Women: Minority and Young Women at Continuing Risk”, 2002.

Although HIV has been found to infect a disproportionately larger number of men compared to women in the past (due to the high rate of transmission through homosexual intercourse among gay men), the rate of HIV infection in women is slowly becoming equal to that seen in men. In 1999, the number of females diagnosed with HIV made up 25% of the total population of individuals infected with HIV, up from the 1985 statistic of 7%. In the U.S., AIDS is now the fifth leading cause of death for all women between the ages of 25-44, and the third leading cause of death for African American women aged 25-44. Id.

While drugs and other methods of treatment are effective at slowing the spread of viral causative agents, they do not prevent transmission of the disease to sexual partners. One of the most effective means of combating the spread of viral STDs, and STDs in general, is through prevention of transmission to uninfected partners. Presently, the only effective means of prevention (barring abstinence) is the use of a physical barrier, such as a condom. However, in some situations it is difficult to negotiate the use of a condom and even when one is used, there still exists the possibility that infection will occur following breaking, leaking or loss of this protective barrier.

Other means to combat the spread of STDs may be through the use of vaccines. Unfortunately, vaccines are very specific to a particular type and/or strain of virus, which significantly limits their applicability. Moreover, viruses are known to mutate and become resistant to vaccines.

Another potentially powerful method for the broad spectrum prevention of sexual transmission of a large variety of pathogens is provided by topically applied microbicidal formulations. “Recommendations for the development of vaginal microbicides,” International Working Group on Vaginal Microbicides, AIDS, 10:1-6 (1996). Microbicides can prevent infection by interrupting one of three possible routes of pathogen transmission, and are grouped accordingly into three classes. Hayes et al., “Microbicides in HIV prevention,” BMJ, 322:410-413 (2001).

The first group of compounds interacts with and disrupts the plasma membrane/viral envelope of the infectious agent thereby disabling or destroying the pathogen. These compounds include surfactants, peptides, plant extracts, and acid buffers. The second group includes compounds that directly inhibit viral entry by binding to receptors on the surface of host cells. These include the naphthalene sulfonate polymer PRO 2000, and sulfonated polysaccharides (including carrageenan and cellulose sulfate). The third class includes compounds that inhibit reverse transcription of the viral genome within the host cell. One compound belonging to this class is the reverse transcriptase inhibitor Tenovir. Unfortunately, compounds that exhibit high efficacy against infectious agents are often found to cause lesions in the vaginal epithelium, leaving women more vulnerable to infection.

It has been previously reported that certain lipids have antimicrobial effects. Kabara, “The Pharmacological Effects of Lipids,” ed. 1987, and Nutritional Biochemistry, Vol. 6, July, 1995. Without being bound by any theory, it is believed that these lipids act by disrupting the infectious organism's lipid envelope or membrane leading to changes in the organism's permeability resulting in loss of infectivity.

Unfortunately, the high lipophilicity makes these antimicrobial lipids insoluble in aqueous solutions, and thus difficult to use them as prophylactic and/or therapeutic agents. The solubility problems can be overcome to some extent through the use of nonaqueous solvents such as ethanol or dimethylsulfoxide (DMSO). However, such solvents, in many instances, are inappropriate for use in humans or animals.

### BRIEF SUMMARY OF THE INVENTION

The present invention overcomes the solubility problem of the active agent, such as antimicrobial lipids, by formulating it using a lipid vesicle. Such a liposomal formulation provides controlled release, target specificity, and/or prolonged life span of the active agent, as well as other advantages.

Preferably, the liposomal formulation comprises a lipid vesicle and at least one single chain lipid active agent, which has antimicrobial activity. Thus, compositions of the present invention are useful in preventing and/or treating a microbial infection in a mammal.

### BRIEF DESCRIPTION OF THE DRAWINGS

FIG. 1 shows histological effect of liposome encapsulated octylglycerol on human vaginal eptithelium. Tissues were exposed to the microbicide at 37° C. for 2 hours. Digital images were captured with a Zeiss microscope using a 10 X objective.

### DETAILED DESCRIPTION OF THE INVENTION

I. Definitions

“Active agent” refers to a pharmaceutically active compound.

“Acyl” refers to a moiety of the formula —C(═O)—R^{a}, wherein R^{a} is an aliphatic hydrocarbon group.

“Alkyl” refers to a linear saturated monovalent hydrocarbon moiety or a branched saturated monovalent hydrocarbon moiety. Exemplary alkyl groups include, but are not limited to, methyl, ethyl, n-propyl, 2-propyl, tert-butyl, pentyl, hexyl, heptyl, octyl, nonyl, decyl, undecyl, dodecyl, and the like.

“Alkenyl” refers to a linear monovalent hydrocarbon moiety or a branched monovalent hydrocarbon moiety having one or more, preferably one or two, carbon-carbon double bonds. Exemplary alkenyl groups include, but are not limited to, ethenyl, propenyl, butenyl, pentenyl, hexenyl, heptenyl, octenyl, nonenyl, decenyl, undecenyl, dodecenyl, pentadienyl, hexadienyl, heptadienyl, octadienyl, nonadienyl, decadienyl, undecadienyl, dodecadienyl, and the like.

“Alkynyl” refers to a linear monovalent hydrocarbon moiety or a branched monovalent hydrocarbon moiety having one or more, preferably one or two, carbon-carbon triple bonds. Exemplary alkynyl groups include, but are not limited to, undecynyl, dodecynyl, pentadiynyl, hexadiynyl, heptadiynyl, octadiynyl, nonadiynyl, decadiynyl, undecadynyl, dodecadiynyl, and the like.

“Aliphatic hydrocarbon” refers to a noncyclic and nonaromatic hydrocarbon moiety having one to twenty four, preferably two to twenty two, more preferably two to sixteen, carbon atoms. Aliphatic hydrocarbon can be linear or branched chain of carbon atoms optionally having one or more, preferably one or two (if present), carbon-carbon double bonds. In addition, one or more hydrogen atoms in the aliphatic hydrocarbon moiety can be replaced with halogen. Thus, the term “aliphatic hydrocarbon” include alkyl, haloalkyl, alkenyl and alkynyl groups. A particularly preferred aliphatic hydrocarbon is an alkyl or haloalkyl group with an alkyl group being an especially preferred aliphatic hydrocarbon moiety.

“Fatty acid” refers to a carboxylic acid composed of a chain of hydrocarbon containing from two to twenty carbon atoms. The hydrocarbon moiety can be saturated or contain one or more carbon-carbon double and/or triple bonds.

The terms “halo,” “halogen” and “halide” are used interchangeably herein and refer to a substituent fluoro, chloro, bromo, or iodo.

“Haloalkyl” means alkyl substituted with one or more same or different halo atoms, e.g., —CH₂Cl, —CF₃, —CH₂CF₃, —CH₂CCl₃, and the like. The term “haloalkyl” also includes those alkyl groups in which all alkyl hydrogen atoms are replaced by halides, i.e., perhaloalkyls, such as trifluoromethyl and the like.

“Microbial” refers to bacteria, virus or protozoa. Thus, the term “anti-microbial” compound refers to a compound that can treat or prevent bacteria infections, viral infections and/or protozoan infections.

“Single chain lipid” refers to any compound having one lipid moiety attached to the compound. Preferably, the lipid moiety is covalently attached to the compound. Exemplary single chain lipids include, but are not limited to, mono-lipid glycerol (i.e., glycerol with one lipid group attached to one of the hydroxy groups of glycerol), fatty acids, ethers and esters, lysophospholipids and the like.

II. Liposomal Formulations

One aspect of the present invention provides liposomal formulations comprising a lipid vesicle and at least one single chain lipid active agent. The active agent comprises a lipid moiety, thus rendering the active agent lipophilic. Accordingly, the active agent is relatively insoluble in aqueous solutions, and therefore it is difficult to use the active agent as a prophylactic and/or therapeutic agent in a mammal. The present invention overcomes this solubility problem by providing the active agent in a liposomal formulation. The liposomal formulations of the present invention also provide controlled release, target specificity, and/or prolonged life span of the active agent, as well as other advantages.

The amount of lipid vesicle generally ranges from about 0.01% to about 30% by weight of the total liposomal formulation. A particularly preferred amount of lipid vesicle in liquid drops or spray formulations is from about 0.01 to about 1% by weight of the total liposomal formulation. A particularly preferred amount of lipid vesicle in lotion formulations is from about 0.5% to about 5% by weight of the total liposomal formulation. A particularly preferred amount of lipid vesicle in cream formulations is from about 3% to about 20% by weight of the total liposomal formulation, with the range of from about 5% to about 15% by weight of the lipid vesicle being an especially preferred range. A particularly preferred amount of lipid vesicle in paste formulations is from about 15% to about 50% by weight of the total liposomal formulation.

The amount of active agent is typically from about 0.01% to about 10% by weight of the total liposomal formulation. Preferably, the amount of active agent is from about 0.02% to about 5% of the total liposomal formulation, with the range from 0.05% to about 3% being a particularly preferred amount.

Alternatively, the % weight ratio between the lipid vesicle and the active agent is typically between 1:1 to about 100:1. A particularly preferred % weight ratio between the lipid vesicle and the active agent in the liposomal formulations of the present invention is from about 1:1 to about 50:1. A more preferred ratio between the lipid vesicle and the active agent in the formulation is from about 2:1 to about 25:1, with the ratio range of about 5:1 to about 15:1 being an especially preferred weight % ratio.

A. Lipid vesicle

In one embodiment, the lipid vesicle is comprised of a phospholipid. Exemplary phospholipids that are suitable in compositions of the present invention include lecithin (phosphatidylcholine, i.e., PC), phosphatidylethanolamine (PE), Phosphatidylserine (PS), phosphatidylglycerol (PG), and other appropriate membrane forming lipids known to one skilled in the art (Israelachvili,J., Mitchell,D., and Ninham,B. (1976). Theory of Self-assembly of Hydrocarbon Amphiphiles into Micelles and Bilayers. J. Chem. Soc. Trans. II 72, 1525-1568). A particularly preferred phospholipid is lecithin (phosphatidylcholine). The lipid vesicle can be unilamellar, multilamellar or oligolamellar.

Preferably, the active agent is encapsulated within the lipid vesicle. Encapsulation of the active agent within the lipid vesicle (i.e., liposome) provides enhanced bioavailability by providing a reservoir of stable active agents. In addition, encapsulation of the active agent increases the stability of the active agent and avoids problems associated with insolubility of the lipophilic active agents in aqueous solutions. Liposomes are able to encapsulate lipophilic compounds in their membranes and inter alia, protect them from the aqueous environment. In addition, they can be formulated into a variety of formulations, such as a cream, a lotion, or an aqueous suspension. Moreover, since the liposomes are comprised of natural phospholipids, they are inherently safe and substantially nonimmunogenic.

In another embodiment, liposomal formulations of the present invention further include a co-lipid. A particularly preferred co-lipid is selected from a cholesterol, a second phospholipid, a cationic lipid, an anionic lipid, and a combination thereof. A particularly preferred cationic lipid is selected from the group consisting of stearyl-amine, DC-Chol, DOTAP, and a combination thereof, whereas a particularly preferred anionic lipid is selected from PS, PG, and a combination thereof.

B. Active Agent

The active agents of the present invention are single chain lipids. Typically, the single chain lipids comprise a lipophilic moiety and a hydrophilic moiety. The hydrophilic moiety can comprise one or more hydrophilic groups that can form a hydrogen bond with water. Typically, the hydrophilic moiety comprises one or more heteroatoms each of which is independently selected from O, N, P and S. Exemplary hydrophilic groups that are suitable in the present invention include, but are not limited to, carboxylic acids, esters, amides, carbamates, urea, phosphonic acid, phosphinic acid, phosphate esters, phosphinate esters, ethers, alcohol (i.e., hydroxyl), amines, thiol, sulfinyl, sulfanyl, sulfoxide, and the like, including salt derivatives thereof. Thus, a wide variety of active agents are within the scope of the present invention.

In one particular embodiment, the lipophilic moiety of the active agent is linked to the hydrophilic moiety through an oxygen atom. The linkage can be an ester, ether or a phosphate ester linkage. A particularly preferred linkage is an ether or an ester linkage. An especially preferred linkage between the lipophilic moiety and the hydrophilic moiety of the active agent is an ether linkage.

In another embodiment, the active agent is selected from a monoglyceride, a fatty acid, a lysophopholipid, and a combination thereof. The term “monoglyceride” refers to a mono-lipid glycerol compound, i.e., glycerol with one lipid group attached to one of the hydroxy groups of glycerol. The lipid group can be attached to glycerol through an ester (i.e., monoacylglyceride), ether (i.e., monoetherglycerol) or a phosphate (i.e., monophosphate-glycerol) linkage or a derivative thereof, such as phosphinic or carbonate linkage, with the ester group being a preferred linkage.

A particularly preferred active agent is a monoglyceride. An especially preferred active agent is monoglyceride in which the lipid group is a fatty acid (i.e., fatty acid monoglyceride or monoacylglyceride) or an ether derivative thereof, in which the ester moiety is replaced with an ether group. Within this group of active agents, fatty acid monoglycerides having from about 2 to about 18 number of carbon atoms in the fatty acid moiety are preferred. More preferred fatty acid monoglycerides are those having from about 6 to about 12 number of carbon atoms in the fatty acid moiety. An especially preferred fatty acid monoglyceride is of the formula: 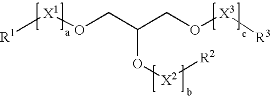
wherein one of R¹, R² and R³ is C₂-C₂₂ aliphatic hydrocarbon, preferably alkyl or haloalkyl, more preferably alkyl, and the other two groups are independently hydrogen, lower alkyl (i.e., C₁-C₄ alkyl) or a hydroxy protecting group; each of the subscripts a, b and c is independently 0 or 1; and each of X¹, X², X³ is a functional group selected from —C(═O)—, —S(O)ₙ— (wherein n is 0, 1 or 2), —OP(═O)_{d}(OR)ₑ— (wherein d is 0 or 1; e is 1 or 3, provided e is 1 when d is 1; and each R is independently hydrogen or alkyl).

In one particular embodiment, a, b and c are 0.

In another embodiment, one of a, b and c is 1 and the others are 0.

When present, a particularly preferred, X¹, X² and X³ is 13 C(═O)—.

Yet in another embodiment, one of R¹, R² and R³ is C₂-C₁₈ alkyl or haloalkyl with the alkyl group being particularly preferred. A particularly preferred compound is one in which one of R¹, R² and R³ is C₆-C₁₂ alkyl or haloalkyl with the alkyl group being especially preferred.

Still further, combinations of the preferred groups described herein form other preferred embodiments. For example, in one particularly preferred embodiment a, b and c are 0, R¹ is C₂-C₁₈ alkyl or haloalkyl, R² and R³ are hydrogen. In this manner, a variety of preferred active agents are embodied within the present invention. For example, a particularly preferred fatty acid monoglyceride is one in which a, b and c are 0, and one of R¹, R² and R³ is C₂-C₁₈ alkyl and the others are hydrogen.

Particularly preferred fatty acid monoglycerides are those wherein one of R¹, R² and R³ is C₈, C₁₀ or C₁₂ alkyl group. Especially preferred fatty acid monoglycerides include 1-O-octyl-sn-glycerol and 2-O-octyl-sn-glycerol, structures of which are shown below. 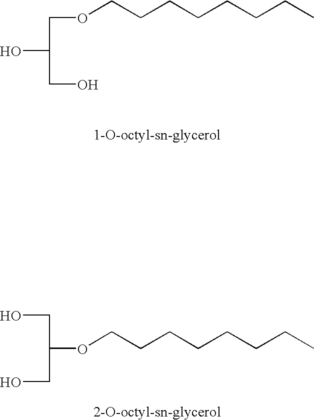
C. Other Agents

Liposomal formulations can also include one or more nontoxic physiologically tolerable or acceptable diluents, carriers, adjuvants or excipient that are collectively referred to herein as excipients. Suitable excipients include antioxidants, co-solvents, preservatives, flavoring agents, vitamins, thickening agents, buffers, wetting agents, emulsifying and suspending agents, sweetening, flavoring and perfuming agents, and the like. Other acceptable excipients are well known to one skilled in the art.

In one particular embodiment, liposomal formulations of the present invention further comprise an excipient selected from a co-solvent, such as propylene glycol and ethanol; an anti-oxidant, such as vitamin E acetate; a preservative, such as methylparaben or propylparaben; a thickening agent, such as Carbopol, Crothix, a buffer, and a combination thereof.

In some embodiments, liposomal formulations of the present invention can also comprise physiologically acceptable sterile aqueous or nonaqueous solutions, dispersions, suspensions or emulsions and sterile powders for reconstitution into sterile injectable solutions or dispersions. Examples of suitable aqueous and nonaqueous carriers, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the maintenance of the required particle size in the case of dispersions and by the use of surfactants. It may also be desirable to include isotonic agents, for example sugars, sodium chloride and the like.

Liposomal formulations of the present invention can be administered to mammals (e.g., humans and other animals) either orally, parenterally, topically, as a paste, a cream, a lotion a spray, or as a suppository. As such, liposomal formulations of the present invention can be formulated in a wide variety of formulations known to one skilled in the art, such as an oral formulation, a parenteral formulation, a topical formulation including, mucosal (buccal, vaginal, rectal) formulations, dermal formulations, nasal formulations, and an ophthalmic formulation. Liposomal formulations of the present invention are preferably administered in a form and via a route that delivers them most directly to the site of possible infection site.

The liposomal formulations of the present invention can be administered to a patient to prevent a variety of microbial infections. Liposomal formulations are generally administered in a variety of forms adapted to the chosen route of administration, e.g., orally or parenterally. Parenteral administration in this respect includes, but is not limited to, administration by the following routes: intravenous; intraperitoneal; intramuscular; subcutaneous; intraocular; intrasynovial; transepithelially including transdermal. Topical administration in this respect includes, but is not limited to, administration by the following routes ophthalmic, sublingual, buccal, dermal, rectal, vaginal, and nasal or intratracheal inhalation via insufflation of an aerosol.

In one embodiment, liposomal formulations of the present invention are formulated as a topical formulation. A particularly preferred liposomal formulation is selected from cream, a gel, a lotion, a suppository, a fluid suspension, and a paste. Typically, liposomal formulations of the present invention are formulated into liposomal creams to enhance bioavailability of the active agents. Liposomes are especially suited for this task since they are composed of natural phospholipids and are, therefore, relatively nontoxic.

The liposomal formulations can be orally administered, for example, with an inert diluent or with an assimilable edible carrier, or it can be enclosed in hard or soft shell gelatin capsules, or it can be compressed into tablets, or it can be incorporated directly with the food of the diet. For oral therapeutic administration, the liposomal formulation can be incorporated with excipient and used in the form of ingestible tablets, buccal tablets, troches, capsules, elixirs, suspensions, syrups, wafers, and the like. Such compositions and preparation can contain at least 0.1% of active agent. The percentage of the liposomal formulation and preparation can, of course, be varied and can conveniently be between about 1 to about 30% of the weight of the total composition. The amount of liposomal formulation in such therapeutically useful compositions is such that a suitable dosage of the active agent will be obtained. Preferred compositions or preparations according to the present invention are prepared such that an oral dosage unit form contains from about 1 to about 1000 mg of liposomal formulations.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the liposomal formulations, sucrose as a sweetening agent, methyl and propylparabens as preservatives, a dye and flavoring such as cherry or orange flavor. Of course, any material used in preparing any dosage unit form should be pharmaceutically pure and substantially nontoxic in the amounts employed. In addition, the liposomal formulation can be incorporated into sustained-release preparations and formulation.

The liposomal formulations can also be administered parenterally. Solutions of the liposomal formulations can be prepared in water suitably mixed with a surfactant such as hydroxypropylcellulose. Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacteria and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various other antibacterial and antifungal agents, for example, parabens, chlorobutanol, phenol, sorbic acid, thimerosal, and the like. In some embodiments, isotonic agent(s), e.g., sugars and/or sodium chloride, are also included.

Sterile injectable solutions can be prepared by incorporating the liposomal formulation in the required amount in the appropriate solvent with various other ingredients enumerated above, as required, optionally followed by filtered sterilization. Generally, dispersions are prepared by incorporating the various sterilized liposomal formulation into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the liposomal formulation plus any additional desired ingredient from previously sterile-filtered solution thereof.

The liposomal formulations of the present invention can be administered to a mammal alone or in combination with pharmaceutically acceptable carriers, as noted above, the proportion of which is determined by the solubility and chemical nature of the liposomal formulation, chosen route of administration and standard pharmaceutical practice.

III. Liposome Preparation

Various methods of liposome and liposome-like preparations as potential drug carriers have been reviewed (see, e.g., U.S. Pat. Nos. 5,567,434, 5,552,157, 5,565,213, 5,738,868 and 5,795,587, each specifically incorporated herein by reference in its entirety).

In a preferred embodiment, the lipid formulations of the present invention are produced using the apparatus and method described in PCT Publication No. WO 00/29103, published on May 25, 2000, and incorporated herein by reference.

An apparatus is described therein that is useful for the continuous production of a composition of matter by in-line mixing. The apparatus comprises a first phase storage means capable of being maintained at a set temperature and a first pressurized transfer means for transferring the first phase from the storage means, along with an second phase storage means capable of being maintained at a set temperature and a second pressurized transfer means for transferring the second phase from the storage means. As described therein, the first phase is a lipid phase (optionally containing an active agent) and the second phase is an aqueous phase. The lipid phase storage means is capable of being maintained at a set temperature by a first temperature control means, typically within the range of about 20 to 75° C. Similarly, the aqueous phase storage means is capable of being maintained at a set temperature by a second temperature control means, typically within the range of about 20 to 75° C. The lipid phase and aqueous phase storage means are equipped with a means for continuously replenishing the lipid and aqueous phases. In this manner, the storage means function as a temperature stabilization means such that the lipid and aqueous phases are continuously fed into the storage means, where the temperature of each phase is stabilized prior to introduction into pressurized transfer means that exits each respective storage vessel.

The apparatus also has a mixing device that comprises a first metering system for receiving the lipid phase from the first pressurized transfer means, a second metering system for receiving the aqueous phase from the second pressurized transfer means, a pre-mixing system for preparing a pre-mixed formulation, a third pressurized transfer means for transferring the lipid phase from the first metering system to a first inlet orifice in the pre-mixing system and a fourth pressurized transfer means for transferring the aqueous phase from the second metering system to a second inlet orifice in the pre-mixing system. The pre-mixing system comprises a pre-mixing chamber having a first and second inlet orifice. The pre-mixing system can further comprise a means for creating turbulence in the aqueous phase prior to entry into the pre-mixing chamber.

The apparatus also has a mixer such as a static mixer for preparing a mixed formulation comprising lipid vesicles, having a mixing chamber and an optional means for determining the optical properties of the mixed formulation, a fifth pressurized transfer means for transferring the pre-mixed formulation from the outlet orifice of the pre-mixing system to the mixing chamber or other suitable connection or fitting; and an optional means for applying ultrasonic energy to the pre-mixing system, the mixing chamber or both. In a preferred embodiment, the optical properties of the mixed formulations are measured, with the means for determining the optical properties of the mixed formulation being configured so as to control the first and second temperature control means and the first and second metering systems.

The apparatus and method of the invention provide for lipid phase and aqueous phase streams that are as pulse-less as possible and are maintained at a constant pressure. This is achieved by the precise metering systems each of which is provided with a pump that operates under positive pressure and in such a manner so as to provide precise volumetric delivery.

The mixer is preferably a static mixer, such as a laminar division type inline mixer. The mixer may have a means for controlling the temperature of the mixing chamber, which is typically within the range of about 20 to 80° C. In addition, the mixer may also have a means for controlling the degree and rate of mixing within the mixing chamber. The mixing device of the apparatus may also have a means for controlling the temperature within the open space of the mixing device, which is also typically within the range of about 20 to 80° C.

The apparatus has a dispensing means for transferring the mixed formulation from the mixing chamber into a storage chamber. This apparatus is particularly useful for the production of lipid vesicles, and more particularly multilamellar lipid vesicles. The apparatus of the invention is readily evaluated as to its particular suitability for manufacturing lipid vesicles having a pre-specified composition and configuration.

In general, liposomes are formed from phospholipids that are dispersed in an aqueous medium and spontaneously form multilamellar concentric bilayer vesicles (also termed multilamellar vesicles (MLVs). MLVs generally have diameters of from 25 nm to 10 μm. Sonication or microfluidization of MLVs results in the formation of small unilamellar vesicles (SUVs) with diameters in the range of 200 to 2,000 Å, containing an aqueous solution in the core.

IV. Utility

Active agents that are present in liposomal formulations of the present invention have antimicrobial activity. Accordingly, liposomal formulations of the present invention can be used to inhibit microbial infections. As used herein, the term “inhibit” means to treat or prevent. In particular, active agent component of liposomal formulations of the present invention has antimicrobial and antiviral activity. Thus, liposomal formulations of the present invention are particularly useful in preventing and/or treating bacterial, viral and protozoan infections.

As stated above, liposomal formulations of the present invention enhance water solubility and/or stability of the active agents. Thus, liposomal formulations of the present invention enhance bioavailability and/or stability of the active agents.

Another aspect of the present invention provides a process of inhibiting an infection caused by a pathogenic microorganism in a subject in need of such treatment. In accordance with that process, the subject is administered a pharmaceutically sufficient amount of the liposomal formulation of the present invention to effectively inhibit the infectious activity of pathogenic microorganisms. A preferred subject is a human. A pathogenic microorganism can be a bacteria, a virus or a parasitic protozoa. The active agents present in liposomal formulation of the present invention are shown herein to be particularly effective in inhibiting sexually transmitted infections.

Active agents of the present invention are an effective virucide against infections caused by a wide range of viruses. In one particularly embodiment, formulations of the present invention are useful in treating and/or preventing infections caused by enveloped viruses. Exemplary enveloped viruses include, but are not limited to, Herpes Simplex Virus (HSV) Type 1 and Type 2, Vesicular Stomatitis Virus (VSV), Visna Virus (VV), Measles Virus (MV), and Human Immunodeficiency Virus (HIV). Accordingly, in one particular embodiment, liposomal formulations of the present invention are useful in preventing a viral infection, in particular a viral infection that is selected from VSV, VV, MV, HSV, and HIV.

Active agents of the present invention are also effective bactericides against infections caused by a wide range of bacteria including, but not limited to, gram-positive and gram-negative bacteria, including those that cause sexually transmitted diseases (i.e., STD's), such as gonorrhea and chlamydia. Exemplary bacteria infections that can be prevented include, but are not limited to, members of the genus Streptococcus, Haemophilus, Helicobacter, Staphylococcus, Enterococcus, Micrococcus, Enterobacter, Klebsiella, Providensia, Pseudomonas, Acinetobacter, Candida, Mycobacterium, Nocardia, Escherica, Salmonella, and Chlamydia. Exemplary particular bacteria are S. aureus, S. epidermidis, S. bovis, S. agalactiae, S. pyogenes, M. luteus, P. aeruginosa, M. smegmatis, N. asteroides, S. pneumoniae, H. influenzae, H. pylori, E. coli, Salmonella enteritidis, N. Gonorrhoeae and Chlamydia trachomatis. In particular, liposomal formulations of the present invention are useful in preventing gonorrhea and chlamydia.

Additional objects, advantages, and novel features of this invention will become apparent to those skilled in the art upon examination of the following examples thereof, which are not intended to be limiting.

### EXAMPLES

### Example 1

This example illustrates effect of lipid concentration.

Liposome formulations containing 0.5 wt % octylglycerol were prepared using 1, 2.5, 5, and 10 wt % phosphatidylcholine (PC). The anti-microbial activity of the formulations was tested on several strains of gonorrhea, HSV-1, HSV-2, and HIV. In addition, the formulations were tested for safety towards naturally occurring vaginal flora, such as Lactobacillus. Formulations without OG served as negative controls. HSV-1 and HSV-2 were incubated with each formulation (at a ten fold dilution) for 30 minutes at 37° C. and then assayed for remaining viral infectivity in a cell culture system. Infectivity remaining after exposure to each liposome formulation was expressed as a TCID₅₀ (50% Tissue culture infectious dose). Formulations considered effective reduced viral infectivity by at least 1,000 fold.

All octylglycerol containing formulations were found to effectively kill all strains of gonococcus tested as well as HSV-1 and HSV-2. See Table I below. Infection of PHA-stimulated PBMCs by HIV appeared to be suppressed by all liposome-encapsulated octylglycerol formulations (Table I), but not by the controls. It was found that 1% PC+0.5% OG suppressed about 90% of the HIV infection as compared to 10% PC+0.5% OG, which suppressed only 80% of HIV infection. This suggests an inverse correlation between the amount of lipid and the activity of the formulation. Without being bound by any theory, it is believed that this reduced activity at higher lipid concentration is due to less active agent being present per liposome. None of the control formulations without OG appeared to have any effect on gonococcus, HIV or HSV. This shows the activity was due to OG and not due to the delivery vehicle.

The phospholipid concentration has no apparent effect on the ability of OG-containing formulations to reduce gonorrhea, HSV-1 or HSV-2. Octylglycerol-encapsulated liposomes do not appear to be toxic to Lactobacillus.

TABLE I
Antimicrobial activity of liposome-encapsulated 0.5 wt % OG
in relation to PC content. The “+” indicates a reduction
in infectivity of at least 1,000 fold. In case of HIV, the % inhibition
of infection is indicated. None of the controls formulations without
OG displayed any killing or inhibition of infection.

| | PC (wt %) | | | |
|---|---|---|---|---|
| Infectious Agent | 1 | 2.5 | 5 | 10 |
| Gonococcus (Strain 1: ATCC19425) | + | + | + | + |
| Gonococcus (Strain 2: GG131) | + | + | + | + |
| Gonococcus (Strain 3: DOD633) | + | + | + | + |
| Gonococcus (Strain 4: UPS1170) | + | + | + | + |
| Gonococcus (Strain 5: ATCC49226) | + | + | + | + |
| HSV Type 1 | + | + | + | + |
| HSV Type 2 | + | + | + | + |
| HIV (test 1) | 91% | 87% | 86% | 80% |
| HIV (test 2) | 90% | 87% | 87% | 80% |
| Lactobacillus | − | − | − | − |

### Example 2

This example shows effects of octylglycerol dose.

The concentration of octylglycerol which was most effective at killing infectious organisms was determined using formulations containing 10% PC and 0.1, 0.3, 1, 3, and 6% octylglycerol, in citrate buffer pH 5. In addition, the formulations contained the preservatives methylparaben (0.046 wt %) and propylparaben (0.02 wt %). These preservatives had previously been shown to be nontoxic to Lactobacillus. The 10% PC formulation was chosen because it was a pharmaceutically elegant formulation. It had a viscosity similar to that found in commercially available products for vaginal use (i.e. lubricants, spermicides). These formulations were tested on the five gonococci strains, HSV-1, HSV-2, and HIV. In addition the safety of these formulations towards Lactobacillus was tested. The results are presented in Table II.

Formulations containing 1-6% OG were effective at killing all strains of gonorrhea tested, HSV-1, HSV-2, and HIV. The killing of HIV appeared to increase with increasing OG concentration. The two highest OG concentrations (3 and 6%) appeared to coincide with an increased toxicity to the mammalian cells used for the HIV test.

TABLE II
Dose response of liposome-encapsulated OG. Liposome
formulations containing 10% PC and various amounts of
OG were tested for antimicrobial activity. The “+” indicates
a reduction in infectivity of at least 1,000 fold. In case of HIV,
the % inhibition of infection is indicated. None of the controls
without OG displayed any killing or inhibition of infection.

| | OG (wt %) | | | | | |
|---|---|---|---|---|---|---|
| Infectious Agent | 0 | 0.1 | 0.3 | 1 | 3 | 6 |
| Gonococcus (Strain 1: ATCC19425) | − | − | − | + | + | + |
| Gonococcus (Strain 2: GC131) | − | − | − | + | + | + |
| Gonococcus (Strain 3: DOD633) | − | − | − | + | + | + |
| Gonococcus (Strain 4: UPS1170) | − | − | − | + | + | + |
| Gonococcus (Strain 5: ATCC49226) | − | − | − | + | + | + |
| HSV Type 1 | − | − | − | + | + | + |
| HSV Type 2 | − | − | − | + | + | + |
| HIV (IIIB) | 8 | 52 | 27 | 90 | 95 | 99 |
| Lactobacillus | − | − | − | − | − | − |

### Example 3

This example illustrates safety of liposomal formulations of the present invention on excised human cervical tissue.

The effect of liposome encapsulated octylglycerol on the integrity of human cervical epithelium was examined ex vivo. Ecto-cervical tissue was obtained from pre-menopausal women undergoing hysterectomy. These tissues were obtained through the Magee Womens Hospital Tissue Procurement Facility as per IRB Protocol MWH-98-065. Tissues were obtained immediately following surgery and release from the pathologist. During transport from pathology the tissue was immersed in media and held at 0-4° C. A piece of each untreated tissue was retained for histological evaluation and served as negative control. Excess stromal tissue was removed. The epithelial layer was isolated using a Thomas-Stadie Riggs tissue slicer and placed in a Franz diffusion cell. The epithelial side of the tissue was oriented toward the donor compartment. The receiver compartment contained 4.8 mL of media. The test liposomal formulation was placed in the donor compartment. Tissues were exposed to the donor formulation at 37° C. for 2 hours. At the conclusion of the exposure period histological evaluation was conducted on the exposed tissues.

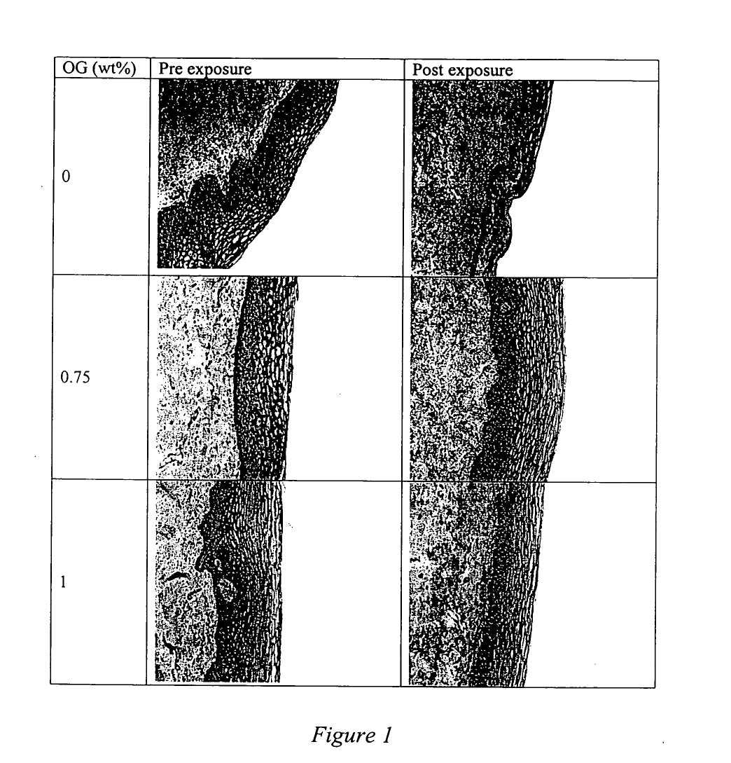

No histological changes in human cervical epithelium could be detected after 2 hours of exposure to 0, 0.75 or 1.0 wt % octylglycerol containing liposomes. See Table III in FIG. 1. Severe shedding of the epithelial layers could be observed when the tissues were exposed to 2% polysorbate 80 (data not shown).

### Example 4

This example illustrates safety of liposomal formulations of the present invention in vivo in a pig-tailed macaque model.

The product was administered rectally at a dose of 1.5 ml daily for three days and effects examined 15 minutes and 24 hours after each application. pH and rectal lavage evaluations were made. These studies show the product to be nondamaging in the rectal pigtailed macaque model.

TABLE III

| Day 1 | | Day 2 | | Day 3 | | Day 4 |
|---|---|---|---|---|---|---|
| T-0 | T-15 | T-0 | T-15 | T-0 | T-15 | T-0 |

No Product

| | | | | | | |
|---|---|---|---|---|---|---|
| 6.8 ± | 7.2 ± | 6.2 ± 0.6 | 6.5 ± 0.0 | 7.3 ± 0.8 | 7.5 ± 0.9 | 7.2 ± 0.3 |
| 0.8 | 1.0 | | | | | |

Liposome encapsulated octylglycerol

| | | | | | | |
|---|---|---|---|---|---|---|
| 7.2 ± | 7.3 ± | 7.2 ± 1.0 | 6.7 ± 0.3 | 7.2 ± 1.0 | 6.5 ± 0.9 | 7.7 ± 0.6 |
| 1.0 | 0.6 | | | | | |

Data are average pH values of 3 monkeys plus/minus standard deviation.
T-0 indicates the sample time was before application of the product.
T15 indicates that the pH was measured 15 minutes after application of the product.

TABLE IV

| Day 1 | | Day 2 | | Day 3 | | Day 4 |
|---|---|---|---|---|---|---|
| T0 | T15 | T0 | T15 | T0 | T15 | T0 |

No Product

| | | | | | | | |
|---|---|---|---|---|---|---|---|
| Epith.^{a} | 0 | 0 | 0 | 0 | 1: 1 | 1: 8 | 1: 2 |
| Stroma^{b} | 0 | 0 | 0 | 0 | 0 | 0 | 0 |
| Blood^{b} | 0 | 0 | 0 | 0 | 1 | 1 | 0 |

Liposome encapsulated octylglycerol

| | | | | | | | |
|---|---|---|---|---|---|---|---|
| Epith.^{a} | 1: 3 | 0 | 1: 2 | 0 | 1: 2 | 1: 1 | 1: 2 |
| Stroma^{b} | 0 | 0 | 0 | 0 | 0 | 0 | 0 |
| Blood^{b} | 0 | 0 | 1 | 0 | 0 | 1 | 0 |

a: # of animals; # of sheets larger than 3 mm/Animal
b: # of Animals
Data are averages of 3 monkeys plus/minus standard deviation.
T0 indicates the sample time was before application of the product.
T15 indicates that the lavage was taken 15 minutes after application of the product.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method for preventing an infection in a mammal, said method comprising: administering a pharmaceutically effective amount of a liposomal formulation to said mammal, wherein said liposomal formulation comprises:
a) a lipid vesicle; and
b) at least one single chain lipid active agent.

2. The method of claim 1, wherein said active agent comprises an aliphatic hydrocarbon moiety.

3. The method of claim 1, wherein said active agent is selected from the group consisting of a monoglyceride, a fatty acid, a lysophospholipid, and a combination thereof.

4. The method of claim 1, wherein said infection is a viral infection.

5. The method of claim 4, wherein said virus is an enveloped virus.

6. The method of claim 5, wherein said virus is selected from the group consisting of VSV, VV, MV, HSV, and HIV.

7. The method of claim 1, wherein said infection is a bacterial infection.

8. The method of claim 7, wherein said bacterial infection is selected from the group consisting of Gonorrhea and Chlamydia.

9. The method of claim 1, wherein said infection is a parasitic protozoan infection.

10. The method of claim 7, wherein said protozoa is Giardia lamblia.

11. The method of claim 1, wherein said formulation is selected from the group consisting of a topical formulation, an oral formulation, a mucosal formulation, a nasal formulation, an ophthalmic formulation, a rectal formulation, vaginal formulation, parenteral formulation and dermal formulation.

12. A liposomal formulation comprising:
a) a lipid vesicle; and
b) at least one single chain lipid active agent.

13. The liposomal formulation of claim 12, wherein said active agent is selected from the group consisting of a monoglyceride, a fatty acid, a lysophospholipid, and a combination thereof.

14. The liposomal formulation of claim 13, wherein said active agent is a monoglyceride.

15. The liposomal formulation of claim 14, wherein said monoglyceride is a monoalkyletherglyceride with a number of carbon atoms in the alkyl moiety portion being from about 2 to about 18.

16. The liposomal formulation of claim 15, wherein said monoglyceride is selected from the group consisting of 1-O-alkyl-sn-glycerol, 2-O-alkyl-sn-glycerol, and a mixture thereof.

17. The liposomal formulation of claim 16, wherein said monoglyceride is 1-O-octyl-sn-glycerol, 2-O-octyl-sn-glycerol, and a mixture thereof.

18. The liposomal formulation of claim 14, wherein said monoglyceride is a single chain fatty acid monoglycerides with a number of carbon atoms in the fatty acid moiety portion being from about 6 and about 12.

19. The liposomal formulation of claim 12, wherein said lipid vesicle comprises a phospholipid.

20. The liposomal formulation of claim 19, wherein said phospholipid is phosphatidylcholine.

21. The liposomal formulation of claim 20, wherein said lipid vesicle further comprises a diluent selected from the group consisting of a co-solvent, a buffer solution, an anti-oxidant, a preservative, a thickening agent and a mixture thereof.

22. The liposomal formulation of claim 21, wherein said co-solvent comprises propylene glycol, ethanol, water or mixtures thereof.

23. The liposomal formulation of claim 21, wherein said anti-oxidant comprises vitamin E acetate.

24. The liposomal formulation of claim 21, wherein said preservative comprises methylparaben, propylparaben or mixtures thereof.

25. The liposomal formulation of claim 21, wherein said thickening agent comprises Carbopol, Crothix or mixtures thereof.

26. The liposomal formulation of claim 12, wherein said lipid vesicle is unilamellar.

27. The liposomal formulation of claim 12, wherein said lipid vesicle is multilamellar.

28. The liposomal formulation of claim 12, wherein said lipid vesicle is oligolamellar.

29. The liposomal formulation of claim 12, wherein said lipid vesicle comprises a co-lipid.

30. The liposomal formulation of claim 29, wherein said co-lipid is selected from the group consisting of a cholesterol, a phospholipid, a cationic lipid, an anionic lipid, and a combination thereof.

31. The liposomal formulation of claim 30, wherein said cationic lipid is selected from the group consisting of stearyl-amine, DC-Chol, DOTAP, and a combination thereof.

32. The liposomal formulation of claim 30, wherein said anionic lipid is selected from the group consisting of PS, PG, and a combination thereof.

33. The liposomal formulation of claim 12, wherein said formulation is a topical formulation.

34. The liposomal formulation of claim 33, wherein said topical formulation is selected from the group consisting of cream, a gel, a lotion, a suppository, a fluid suspension, and a paste.

35. The liposomal formulation of claim 12, wherein said active agent is encapsulated by the lipid vesicle.

36. A pharmaceutical composition comprising:
a pharmaceutical excipient; and
a liposomal formulation comprising a lipid vesicle and at least one single chain lipid active agent.

37. The composition of claim 36, wherein said excipient comprises an antioxidant, a co-solvent, a preservative, a flavoring agent, vitamin, a thickening agent, a buffer solution, a wetting agent, an emulsifying agent, a suspending agent, a sweetening agent, a flavoring agent, a perfuming agent or mixtures thereof.
